(19) United States
(12) Patent Application Publication
(10) Pub. No.: US 2012/0276054 A1
(43) Pub. Date: Nov. 1, 2012

(54) Alternative bacterial treatment

(76) Inventors: Henry N. Williams, Tallahassee, FL (US); Paul A. Gulig, Gainesville, FL (US); Huan Chen, Tallahassee, FL (US)

(21) Appl. No.: 13/066,913

(22) Filed: Apr. 28, 2011

(51) Int. Cl. A61K 35/66; A61P 31/04

(52) U.S. Cl. 424/93.3

(57) ABSTRACT

Alternative treatments are provided for use in the inhibition, decrease, therapeutic or prophylactic support of a Bdellovibrio and like organism (BALO) Gram negative prey bacterial infection in a host.

### GOVERNMENTAL INTEREST

This invention was supported with United States Government funding through the United States Department of Agriculture, Cooperative State Research, Education and Extension Service Program. Contract/Grant No. 2007-35201-18398. The United States government has certain rights in the invention.

### CROSS REFERENCE TO CO-PENDING APPLICATION

### Background

Bacterial infections afflict multicellular organisms, such as, for example, plants, animals, amphibians and humans at various stages of development. Among children, the elderly and other immunocompromised individuals, bacterial infections can be especially dangerous. Ear, sinus, skin, wound, food-borne and respiratory infections are relatively common, especially in children and are among the leading cause of visits by children to physicians. In circumstances where an individual suffers from chronic bacterial infections and are given conventional antibacterial medicaments, he or she may be at greater risk of developing bacteria that are resistant to the conventional medicaments. In fact, bacteria that are prone to becoming resistant to antibiotics are known to cause ear infections, nose, throat and skin infections.

Therefore, there is a need for novel medications, therapies, methods and approaches to treating bacterial infections.

Many diseases are caused by bacterial infections. Bacteria cause up to twenty-five percent of upper respiratory tract infections. Streptocci bacterial are responsible for almost all cases of strep throat in the United States. Otitis media or middle ear infections are the most common bacterial infection in children in the United States. By age three, two-thirds of children in the United States have had at least one episode of ear infection and the other one third has had three or more episodes. Common lower respiratory tract infections caused by bacteria include pneumonia and bronchitis. Tuberculosis is another common bacterial infection afflicting over 10 million people in the United States.

Infectious diarrhea continues to be a leading cause of morbidity and mortality in the world. Most causes of diarrhea are viral in origin; however, bacteria remain an important cause of diarrhea. Other bacterial infections include salmonella, shigella, Escherichia coli, and skin infections. Bacterial skin infections include impetigo, boils, carbuncles, cellulitis, and burn complications.

The present invention relates to antimicrobial agents and methods that have, for example, prophylactic and inhibitory activity with respect to bacterial infections. The invention also relates to the use of such antimicrobial agents and methods to inhibit, control, provide prophylactic support or treat diseases and disorders related to bacterial infections. More particularly, the present invention provides alternative treatments using alone or in combination live, and or dead antibacterial agents for use, for example, in treating bacterial infections. Even more particularly, the present invention relates to the inhibition, therapeutic and prophylactic preparations comprising certain isolates of the bacterial group Bdellovibrio and like organism (BALO) in combinations of various percentages with certain viruses that kill bacteria (bacteriophages). In particular, the invention provides in one aspect, prophylactic and therapeutic compositions comprising certain Bdellovibrio and like organism (BALO) strains, acting in combination with bacteriophage against, for example, infections of animals and humans caused by some Gram negative pathogenic bacteria including, for example Acinetobacter calcoaceficus, Aeromonas hydrophila, Enterobacter aerogenes, Escherichia coli ML-35, Pseudomonas putida, Pseudomonas spp, Proteus mirabilis, Providencia stuartii, Salmonella, Salmonella Michigan, Salmonella Gaminola Salmonella Montbidea, Salmonella Poona, Vibrio 01, Vibrio vulnificus CMCP6, Vibrio vulnificus M06, Vibrio sp., Vibrio parahaemolyticus P5, and the like. The foregoing needs are met, to an extent, by the present invention, wherein in one aspect a method is provided of inhibiting, decreasing , treating, providing prophylactic support, or preventing a bacterial infection in a host subject comprising administering a therapeutically effective amount of a live Bdellovibrio and like organism (BALO) and an active bacteriophage to a Gram-negative bacteria-infected host subject. The Bdellovibrio and like organism (BALO) may comprise, for example Bdellovibrio bacteriovorus, Bacteriovorax stolpii, Bacteriovorax starrii, Bacteriovorax marinus, Bdellovibrio bacteriovorus HD 100, Bdellovibrio bacteriovorus BD-610, Bacteriovorax Phylotype X, Bacteriovorax Phylotype IX, Bacteriovorax Phylotype V, Bacteriovorax Phylotope III, Bacteriovorax litoralis, and the like.

The BALO Gram negative prey bacteria may comprise Acinetobacter calcoaceficus, Aeromonas hydrophila, Enterobacter aerogenes, Escherichia coil ML-35, Pseudomonas putida, Pseudomonas spp, Proteus mirabilis, Providencia stuarlii, Salmonella, Salmonella Michigan, Salmonella Gaminola Salmonella Montbidea, Salmonella Poona, Vibrio 01, Vibrio s CMP6, Vibrio vulnificus M06, Vibrio sp., Vibrio parahaemolyticus P5 and the like. Prophylaxis or treatment may also be achieved by including in the preparation a number of, for example, Bdellovibrio and like organisms (BALO's) in various combinations with bacteriophage strains, each having different specificities for the target prey bacteria giving the preparation an overall total effectiveness against many more strains than the individual bacteriophages or individual BALO. The phage panel may include one or more bacteriophage strains which are effective against a broad spectrum of bacteria so that the bacteriophages in the composed preparation have overlapping effectiveness, with some specific bacteria being targeted by multiple bacteriophages, thus helping to minimize any development of resistance. Individual strains of the target bacterial species may therefore be killed by one or more of bacteriophages making up a composition or preparation.

The bacteriophage strains may comprise, for example, CK2, 3a, ap3, A3/2, A10/A45, B9PP, A36, E13, E14, B9GP, 531, PM6, phage 1, VD13, phage 182, VD1884, λ phage, T even phages, T1UV, T3, T5, T7, Esc-7-11, E920g, pt1 M13, MS2, P1, P2, Phi x 174, Phi 6, λvir, RB69, N4, 121Q, β4Q, HK243, BW-1, Haiti phage, I2-2, PR64FS, phage M, phage J, PR772, C-1, Phi 92, pilHα, Her252, Mu, H-19J, P1kc, R17, omega8, O103 , K20, SS4, K30, O9-1, HK97, P1D, TC4, MB4, MS2, 13/3a, gh-1, epsilon15, phage x, phage 1-11 heidelberg, phage 16-19, phage 7-11, 9266Q, phage 2.5A, phage Jersey, phage Beccles, SasL4, Sas L6, phage O1, Vil, Vill, alpha3A, Beta, phage 16, phage 24, phage X29, Kappa, phage 4996, phage 57, phage e4, phage 45, phage 13, phage 14, phage 32, CP-T1, phage 493, VP1, VP11, VP12, VP6, KVP20, KVP40, Vf33, Phi 16, Phi HAWI-5, Phi PEL8C-1, V71A-6 NCIMB 41174, NCIMB 41175, NCIMB 41176, NCIMB 41177, NCIMB 41178, NCIMB 41179, and the like.

Further examples of bacteriophage strains useful against Vibrio vulnificus include, for example, 152A-2, 152A-8, 152A-9 152A-10, 153A-5, 153A-7, 153A-8, 154A-8, 154A-9, 108A-9, 110A-7, and 7-8a. Phage CK-2 was isolated from estuarine mud sediment. Phage CB1 was isolated from mud sediment, and phage EJc was isolated from oysters together with Phages 1a, 2a, 3a, 4a, 4b, AOIA-D, CKIA-B, and CKIF-G. Bacteriophages, SSP5 and SSP6 which were classified as members of the myoviridae and Siphoviridae families respectively have been reported to have potential to control Salmonella.

The strain of BALO may, for example comprise Bdellovibrio bacteriovorus, Bacteriovorax stolpii, Bacteriovorax starrii, Bacteriovorax marinus, Bacteriolyticum stolpii, Peredibacter starrii, Bdellovibrio bacteriovorus HD 100, Bdellovibrio acteriovorus BD-610, Bacteriovorax Phylotype X, Bacteriovorax Phylotype IX, Bacteriovorax Phylotype V, Bacteriovorax Phylotope III, Bacteriovorax litoralis and the like. The strains included in the preparation may, for example be different strains.

The combination of BALO and bacteriophage may exist in various concentrations, percentages, and ratios. The combination may also exist in equal ratios.

The bacterial infection may comprise a Gram negative bacterium in plants, animals, fish, amphibians, mammals, or for example humans. Bacterial infections may comprise, for example, sinus, respiratory, renal, skin, wounds, or ear infections of the outer or inner ear canal. In the case of such ear infections, the administration of the prophalytic agent or treatment may be accomplished via droplets, creams, injection, oral suspensions, and via other administrations known in the art. Other bacterial infections include, for example, skin infections, burn infections, eye infections, urinary tract, and gastrointestinal infections. Administration of the prophylactic or treatment agent may be accomplished via injection, oral administration, creams, or suspensions, creams or by other means known in the art. Synergistic effects have been found for the combination of Bdellovibrio and like organisms with viruses in the killing of pathogens that may cause these infections.

Unlike antibiotics and antiviral agents that bacteria and viruses respectively can develop resistant to, those pathogens that are susceptible to Bdellovibrio and like Organisms (BALOs) are not known to develop resistance to them. Although some bacteria have been reported to develop resistance to viruses, should this happen the Bdellovibrio and like Organisms (BALOs) should continue to reduce the pathogens but perhaps not as rapidly as the combination with the bacteriophage.

In embodiments, one aspect of the proposed invention shows that the Bdellovibrio and like Organisms (BALOs) and bacteriophage are beneficial as alternative prophylactic or therapeutic agents for reducing Gram-negative bacterial infections in animals and humans. The prey bacteria may comprise, for example, Acinetobactercalcoaceticus, Aeromonas hydrophila, Enterobacter aerogenes, Escherichia coli ML-35, Pseudomonas putida, Pseudomonas spp, Proteus mirabilis, Providencia stuarfii, Salmonella, Salmonella Michigan, Salmonella Gaminola Salmonella Montbidea, Salmonella Poona, Vibro 01, Vibrio s CMP6, Vibrio vulnificus M06, Vibrio sp., Vibrio parahaemolyticus P5 and the like. Achievement of this aspect would introduce a novel and alternative mode of treating some of the major human Gram negative bacterial pathogens such as, for example, Vibrio spp., E. coli, Pseudomonas, Proteus mirabilis, Providencia stuartii, Salmonella, and improve the recovery rate for these diseases. It has also been suggested that periodontal disease therapy could be assisted by the application of Bdellovibrio and like Organisms (BALOs) specifically to reduce the levels of Gram-negative pathogens in the oral cavity.

There has thus been outlined, rather broadly, certain embodiments of the invention in order that the detailed description thereof herein may be better understood, and in order that the present contribution to the art may be better appreciated. There are, of course, additional embodiments of the invention that may be described below and which may form the subject matter of the claims appended hereto. The present invention therefore relates to the fields of medicine, cell biology, mircrobiology biochemistry, and bacteriology.

### DESCRIPTION

### Brief Description of the Drawings

FIG. 1 shows the kinetics of the lysis of prey cells over time in test with Bacteriovorax (Bx) and/or bacteriophages and control (with either BALO predators or no BALO predators). Error Bars are standard error from three independent experiments.

FIG. 2 show the growth dynamics of Bacteriovorax and bacteriophage on prey over a forty hour period as measured by plate counts. Broken lines designate phage count; solid lines represent Bacteriovorax counts. Fl is the flask with both Bacteriovorax and phage. F2 and F3 are the microcosms consisting of vibrio vulnificus with Bacteriovorax and phage respectively.

FIG. 3 shows Transmission Electron Microscopy (TEM) micrographs representing the predation of bacteriophages and Bacteriovorax on prey vibrio vulnificus. The arrow in FIG. 3A points to a prey cell infected by phage CK2. The star indicates a cell infected by Bacteriovorax to form a bdellobast (osmotically stable structure in which the prey cell becomes rounded) and no phage is seen inside. FIG. 3B shows Bacteriovorax and phage both inside a vibrio vulnificus cell. Additional evidence showing Bacteriovorax and phage are able to infect the same cell. The results showed that Bacteriovorax and phage were able to infect vibrio vulnificus cells separately (FIG. 3A & B) and jointly (FIG. 4).

FIG. 4 shows Bacteriovorax and phage infecting vibrio vulnificus cells at the same time. Electron microscopy also confirmed that the Bacteriovorax, was effective at infecting the prey, vibrio vulnificus. Micrographs suggest that competition exists between Bacteriovorax and phages for the source of food as they both are able to prey on the same bacterium. Here we report a novel finding that Bacteriovorax and bacteriophages are able to infect and reside in a single prey cell resulting from dual infection.

### Definitions

“Amount sufficient” as used herein refers to an amount that when placed in contact with a Gram negative bacteria suppresses the growth or reproduction of the bacteria when compared to the absence of the BALO and bacteriophage combination.

“Antibacterial agent” as defined herein refers to an agent or substance that destroys or suppresses the growth or reproduction of bacteria by interfering with protein synthesis, nucleic acid synthesis, or plasma membrane integrity; or by inhibiting critical biosynthetic pathways in the bacteria. Such agents may be administered in concentrations that are safe for the host and can be used as chemotherapeutic agents to prevent or treat bacterial infections.

“Attack Phase” as used herein refers to free-swimming BALO cells propelled by a polar flagellum at high velocities which aids the BALO in efficiently attacking a suitable bacterium that will serve as the substrate for its feeding and growth. The attack phase BALO cell ceases being by the penetration of the BALO cell through the outer membrane layer of the substrate cell and its subsequent loss of mobility.

“Safe and effective amount” as used herein refers to an amount that is effective enough to inhibit, provide prophylactic support, reduce the bacterial cell proliferation, or provide treatment in animals, mammals, and more particularly, in humans without severe side effects.

The term “pharmaceutical carrier” means one or more compatible solid or liquid filler diluents or encapsulation substances which are suitable for administration to those suffering from disease.

“Bacteria” as defined herein refers to a domain of life existing as small unicellular microorganisms that commonly reproduce by cell division (fission) and are contained within a cell wall. They are a natural component of the human body, particularly on the skin, mouth and intestinal tract. Many are beneficial to the environment and living organisms, but some are the cause of many infectious diseases. Infectious bacteria enter the body through torn tissues, the openings of the nose, mouth, lungs, contaminated food, feces, oral fecal contact and can provoke inflammation. “Bacteria” as used herein are neither plants nor animals. Bacteria are living things that belong to a group all by themselves. They are small, single cell organisms called prokaryotes that do not contain a nucleus and are widely distributed in nature, some in very large numbers because they can quickly multiply under suitable conditions. There are many different kinds of bacteria that are separated into different types and groups, each group and sub group having its very own unique qualities. Bacterial can cause myriad diseases. Bacteria that cause disease are called pathogenic bacteria. Bacteria can cause disease in animals, and in plants. Some bacteria attack only one host plant or animal while other bacteria can attack or infect many types of hosts. There is also a great diversity in where bacteria can grow. Some types of bacteria grow best in cool, damp places like in the soil or in a pond while others can grow in hot places like in hot water heaters or near undersea volcanoes. There's even a species of bacteria that can withstand blasts of radiation 1000 greater than would kill a human. No matter where you look, whether on the ground, in your water, or in your stomach, bacteria are there.

“Bacteriophage” (phage) as used herein are obligate intracellular parasites that multiply inside a host bacterium by making use of some or all of the host bio-machinery. A bacteriophage is a virus that is parasitic in bacteria. There are many similarities between bacteriophages and animal cell viruses.

At one time it was thought that the use of bacteriophage might be an effective way to treat bacterial infections, but it soon became apparent that phage were removed from the host body and thus, were thought to be of little clinical value. However, recently, new interest has developed in the possible use of bacteriophage for treatment of bacterial infections and in prophylaxis. In addition, bacteriophage have diagnostic use for the identification of pathogenic bacteria (phage typing). Although phage typing is not used in the routine clinical laboratory, it is used in reference laboratories for epidemiological purposes.

Although different bacteriophages may contain different materials they all contain nucleic acid and protein. Depending upon the phage, the nucleic acid can be either DNA or RNA but not both and it can exist in various forms. The nucleic acids of phages often contain unusual or modified bases. These modified bases protect phage nucleic acid from nucleases that break down host nucleic acids during phage infection. The size of the nucleic acid varies depending upon the phage. The simplest phages only have enough nucleic acid to code for three to five average size gene products while the more complex phages may code for over one hundred gene products.

The number of different kinds of protein and the amount of each kind of protein in the phage particle will vary depending upon the phage. The simplest phages have many copies of only one or two different proteins while more complex phages may have many different kinds. The proteins function in infection and to protect the nucleic acid from nucleases in the environment.

Probably every known bacterium is subject to infection by one or more viruses or “bacteriophages” as they are known. Extensive research concerning phages has been done on the phages that attack E. coli, especially the T-phages and phage lambda.

Like most viruses, bacteriophages typically carry only the genetic information needed for replication of their nucleic acid and synthesis of their protein coats. When phages infect their host cell, the order of business is to replicate their nucleic acid and to produce the protective protein coat. But they cannot do this alone. They require precursors, energy generation and ribosomes supplied by their bacterial host cell.

Most bacteria, specifically pathogenic bacteria, have associated bacteriophages that can take over the bacterial cellular machinery and lyse the bacterial cell to release new phage particles. As such, there was much interest in using “bacteriophage therapy” as a means to treat, prevent and cure infection since their discovery in the early 1900's. However the advent of antibiotics halted the majority of this research until the past decade, which has seen renewed interest in phages due to the increasing problem of antibiotic resistant bacteria. Although phage therapy does show some promise, it has limitations in that bacteria are only susceptible to infection by phage at discrete stages of growth.

Another important issue with regard to bacteriophages is that they can kill large populations of bacteria relatively quickly. This can be important when you are intentionally trying to produce large numbers of bacteria. For example, when cheese is made, we grow bacteria in the milk to give cheese its' distinctive flavor and texture. If the milk gets infected with a bacteriophage, the culture may die out before the cheese fermentation process is completed. When this happens, the cheese manufacturers have vats of partially cultured milk which is not cheese, and which usually has to be thrown away. The cheese manufacturers sometimes refer to this as a vat of cheese that has been “phaged out”; that is, the starter culture to make the cheese has been destroyed by a bacteriophage.

Bdellovibrio and like organism (BALO) Gram negative prey infection host as used herein refers to Gram negative bacterium that serve as viable and susceptible prey or parasitic victims of Bdellovibrio and like organism (BALO), resulting in the Bdellovibrio and like organism (BALO) feeding on the host cell's proteins and nucleic acids and the complete lysis of the invaded host cell and the release of BALO progeny.

“Bdellovibrio growth phase” as referred to herein mean the period when the BALO penetrate through its prey cell wall and begins to feed and grow on its prey. This phase requires the parasitism of a suitable host cell. It is during the Bdellovibrio growth phase when the BALO cells grows inside its prey and multiplies producing new cells. The attacker cells are not especially particular about the prey, except that it must be of the Gram negative type (i.e. having a thin cell wall and characteristic outer membrane.) When the BALO cell enters the host bacterium it dies and bloats into a spherical shape called a bdelloplast. Generally, the host cell loses its structural framework because it is being eaten from the inside. The growing BALO cell is now considered a parasite and continues to elongate into a filament. When the nutrients (proteins, lipids, structural polymers, RNA, and DNA, etc.) are exhausted from the host cell body, the filament partitions into the smaller attack phase cells and are released into the environment.

“Therapeutically Effective Amount” as used herein means an amount useful in healing, prophylaxis or curing certain patients identified in the specification. It is not dependent on the product having an effect in a living being, such as curing disease but, for example, to broadly claim a pharmaceutical composition with a wide range of effects. “Therapeutically Effective Amount” may also be used to represent a pharmaceutical or other composition with a wide range of effects. Those effects do not necessarily include curing diseases in plants, animals, humans, or other species, but may also, for example, comprise amounts for the prevention, inhibition, prophylaxis, or reduction of a condition or disease. The amount may indicate that the claimed pharmaceutical product has utility in the treatment of a disease where such treatment may have an effect on the “healing” or “curing” of the disease in patients within the class covered by the present invention.

The definition of “pharmaceutically acceptable” as used herein is meant to encompass any carrier, which does not interfere with the effectiveness of the biological activity of the active ingredient and that is not toxic to the host to which it is administered.

“Gram positive and Gram negative” as used herein refers to how a bacteria reacts to the Gram stain technique. If it takes the initial stain, it will be purple and be considered Gram positive. If it doesn't take the initial stain, it will be pink and Gram negative. The difference is in the outer casing of the bacteria. A Gram positive bacterium will have a thick layer of peptidoglycan (a sugar-protein shell) that the stain can penetrate. A Gram negative bacterium has an outer membrane covering a thin layer of peptidoglycan on the outside. The outer membrane prevents the initial stain from penetrating.

“Prophylactic treatment”, “prophylactic effects”, “prophylactic support” or “prophylaxis” as used herein, means measures to protect a person from symptoms or conditions of a disease to which he or she has been, or may be expressed.

Bdellovibrio and Like Organisms (Balo's)

The Bdellovibrio and like Organisms are extremely small bacteria with the unique property of being predators of other Gram-negative bacteria. In the presence of viable and susceptible bacteria a BALO cell physically attacks a prey cell, attaches to its surface, penetrates the cell wall, and multiplies within the periplasmic space of its prey. These minute assassins have a peculiar lifestyle: they swim around at high speed and after collision with a susceptible prey bacterium; they attach to their prey and enter its periplasm. BALO and its progeny degrade and consume the cellular constituents. The life cycle of BALO alternates from the mobile, non-growing attack phase and the growth phase. BALO's are found in a wide variety of ecosystems but particularly in sewage and other areas densely populated with bacteria. BALO were not found in frog or crab intestinal tracts, but have been recovered from the gut of humans, horses, and chickens. The occurrence and distribution are influenced by factors such as, for example, temperature, salinity, habitat, prey polulation density, and pollution. Salinity levels have been found, for example, from about 0.0 to over 1.0 percent. Predatory activity of Bdellovibrio has sometimes found to be inhibited by the presence of detergents, heavy metals, and pesticides.

Some Bdellovibrio and like Organisms (BALOs) are halophilic. Halophilic or salt-loving bacteria are indigenous organisms of salt packs, brines, or bodies of salt water. They thrive in concentrations of salt, for example, of from about one percent, to about fifteen percent salt or from about 0.5 to about 2.5 M salt concentration. Salt requirements and salt tolerances of many species vary according to growth conditions such as media composition and temperature. Halophilic bacteria have been found all over the world from the Dead Sea to the Great Salt Lake, and in all of the earth's oceans. They are also found in the saline soils of Antarctica, the tropics and desert environments. Most species maintain their intracellular ionic concentrations at low levels while synthesizing or accumulating organic solutes to provide osmotic equilibrium of the cytoplasm with the surrounding medium.

Bacteriovorax are members of the saltwater genus Bacteriovorax, formerly known as the marine Bdefiovibrio, are obligate predatory bacterium that prey selectively on other Gram-negative bacteria.

Bacteriolyticum stolpii Bacteriolyticum stolpfi is an obligate predatory bacterium that preys upon a wide variety of susceptible Gram-negative bacteria. Bacteriolyticum stolpfi has an optimal growth temperature of from about 29 to about 31° C.

Peredibacter starrii is another strain of the family of Bacteriovoracaceae predatory bacteria. Peredibacter starrii is found in freshwater and in the soil. Peredibacter starrii is vibriod shaped bacteria about 0.4 to about 0.5 μm in length.

Bdellovibrio and Like Organisms (Balos) Method of Attack

Bdellovibrio (Bd), which literally means “curved leech”, and like organisms makes their living by attacking and devouring other bacteria, and are found in diverse environments such as marine and fresh waters, sewage, and soil. Bacteria of this type are characterized by two distinct stages in their life cycle, a predatory “attack” phase, and a parasitic “growth” phase.

During the attack phase, the cell has a curved rod shape of approximately 1.4 micrometers in length, and has a single whip-like projection called a flagellum. This bacterial flagellum rotates like a corkscrew to propel the bacterium at a rate of 100 microns per second. Considering the size of the cell, this corresponds to an incredible 70 body lengths per second! These highly motile attack phase cells have no sense of direction; instead, the flagellum propels the cell in whatever direction it happens to be pointing. Finding prey, therefore, is limited to “bumping” into a suitable prey cell that just happens to be in the right place at the wrong time. When a prey cell is encountered, the BALO continues to rotate and bore its way through the outer cell wall and into the prey where it lodges in the periplasmic space. Once inside, the attacker loses its flagellum, feeds upon the prey cytoplasm, grows and elongates and prepares for the multiplication process.

Prey Species of Bdellovibrio and Like Organisms (Balos)

Acinetobacter calcoaceticus is a pleomorphic aerobic Gram-negative bacillus commonly found in hospital and on hospital patients in the United States and cultured from the patients' sputum or respiratory secretions, wounds, and urine. Acintobacter has been found to colonize irrigating and intravenous solutions. Acinetobacter infections usually involve the respiratory tract, peritoneal fluid, and the urinary tract. Prolonged hospitalization or antibiotic therapy predisposes to Acinetobacter colonization. Acinetobacter have been found to be multi-drug resistant Few antibiotics are active against this organism. Acinetobacter is generally sensitive to Meropenem, Colistin, Polymyxin B, Amikacin, Rifampin, Minocycline, and Tigecycline. Cephalosporins, macrolides, and penicillins have little or no effect on Acinetobacter activity and may predispose to Acinetobacter colonization.

Aeromonas hydrophila (A. hydrophila) is found present in freshwater environments and also in brackish water. The bacterium is capable of causing illness in fish, amphibians and humans. Humans typically acquire infections through an open wound or ingestion of the organism in food or water. Aeromonas hydrophila may cause gastroenteritis in healthy persons or septicemia in persons with compromised immune systems or various malignancies. Aeromonas hydrophila size ranges from about 1.0 to about 3.5 micrometers in length and has a diameter of from about 0.3 to 1.0 micrometers. Aeromonas hydrophila can grow at temperatures of from about 4° C. to about 37° C. with an optimal growth at about 28° C. In humans, Aeromonas hydrophila is typically transmitted through oral fecal contact, contact with contaminated water, food, soil, feces, and ingestion of contaminated fish or reptiles. Most common is infection through an open wound in contaminated water. The microbe is resistant to penicillin, ampicillin, carbenicillin, and ticarcillin but susceptible to cephalosporins, aminoglycosides, carbapenems, chloramphenicol, tetracycline, trimethoprim-sulfamethoxazole, and the quinolones.

Enterobacter aerogenes is a Gram-negative bacillus belonging to the Enterobacteriaceae family. Enterobacter aerogenes is another infection-causing bacteria that is widely distributed in nature, occurring in fresh water, soil, sewage, plants, vegetables, and human feces. Enterobacter aerogenes can cause, for example, burn, wound, skin, endocarditis, soft tissue, ophthalmic, urinary tract infections. It may also be responsible for septicemia and menigitis. Enterobacter aerogenes ranges from about 0.6 to about 1.0 micrometers wide and from about 1.0 to about 3.0 micrometers in length. Optimal growth occurs at from about 35° C. to about 37° C.

Escherichia coli is another prey bacteria for Bdellovibrio and like Organisms (BALOs) which live in the intestines of people and animals. Most varieties of E. coli are harmless or cause brief diarrhea. Some more serious strains can cause severe, bloody diarrhea and abdominal cramps, followed by organ system damage, such as kidney failure. Exposure may come from contaminated water or food such as raw vegetables and under cooked ground beef. Young children and older adults can develop life-threatening kidney failure such as hemolytic uremic syndrome.

Pseudomonas putida are Gram-negative bacteria prey for BALO's. Pseudomonas putida are fluorescent, aeorbic, non spore forming, oxidase positive bacteria. Having one or more polar flagella, they are motile organisms. They can be found in moist environments, such as soil and water, and grow optimally at room temperature. Certain strains have the ability to grow on and break down many dangerous pollutants and aromatic hydrocarbons such as toluene, benzene, and ethylbenzene. Pseudomonas putida can also be used in petroleum plants to purify fuel. Pseudomonas putida play a huge role in bioremediation, or the removal or naturalization of soil or water contaminants. They can degrade toluene, xylene, and benzene, which are all toxic components of gasoline that leak into the soil by accidental spills. Other strains can convert styrene, better known as packing peanuts, which do not degrade naturally, into a biodegradable plastic. Due to the fact that Pseudomonas putida can use styrene as its only source of carbon and energy, it can completely remove this toxic chemical over time from the environment. Pseudomonas putida can also turn Atrizine, a herbicide that is toxic to wildlife, into carbon dioxide and water. Being a non-pathogenic bacterium, there has been only a few instances where Pseudomonas putida has infected humans. For the most part, it has been associated with immunocompromised patients, causing septicaemia, pneumonia, urinary tract infections, nosocomial bacteremia, septic arthritis, or peritonitis. They are able to protect plants from pests, promote plant growth, and clean up organic pollutants found in soil and water.

Pseudomonas spp is a Gram-negative, oxidase-positive, motile rod bacterium which grows yellow-green iridescent colonies. Pseudomonas spp infections can develop in many places on the human body including skin, subcutaneous tissue, bone, ears, eyes, urinary tract, and heart valves. The most serious infections occur in debilitated persons with compromised immune systems resulting from other disease or therapy. Pseudomonas spp occurs most often in hospitals, where it can be found in sinks, antiseptic solutions, and urine receptacles and by cross infection via the hands of hospital personnel. When infection is localized and external, treatment with 1% acetic acid irrigations or topical agents such as polymyxin B or colistin is sometimes effective. Other antibiotics used include amikacin, tobramycin and gentamicin. Several penicillins, including carbenicillin, ticarcillin, piperacillin, mezlocillin, and azlocillin, are active against Pseudomonas.

Proteus mirabilis is part of the normal flora of the human gastrointestinal tract. It can also be found free living in water and soil. When this organism, however, enters the urinary tract, wounds, or the lungs it can become pathogenic. Proteus mirabilis commonly causes urinary tract infections and the formation of stones. Proteus mirabilis is characterized by its swarming motility, its ability to ferment maltose, and its inability to ferment lactose. Proteus mirabilis has the ability to elongate itself and secrete a polysaccharide when in contact with solid surfaces, making it extremely motile on items such as medical equipment. The most common infection involving Proteus mirabilis occurs when the bacterium moves to the urethra and urinary bladder. Although Proteus mirabilis is mostly known to cause urinary tract infections, the majority of urinary tract infections are due to E. coll. Urinary tract infections caused by P. mirabilis occur usually in patients under long-term catherization. The bacteria have been found to move and create encrustations on the urinary catheters. The encrustations cause the catheter to block. Symptoms for urethritis are mild including frequency of urination and pyuria (presence of white blob cells in the urine). Cystitis (bladder infection) symptoms are easier to distinguish and include back pain, concentrated appearance, urgency, hematuria (presence of red blood cells in the urine), and suprapubic pain as well as increased frequency of urination and pyuria.

Pyelonephritis (kidney infection) can occur when the bacteria migrates from the lower urinary tract. Although it is seen as a furtherance of infections, not all patients have the symptoms associated with urethritis and cystitis. Pyelonephritis is marked by nausea and vomiting.

Proteus mirabilis also can enter the bloodstream through wounds. This happens with contact between the wound and an infected surface. The bacteria induce inflammatory response that can cause sepsis and Systemic Inflammatory Response Syndrome (SIRS). SIRS has a mortality rate between twenty and fifty percent.

Proteus mirabilis can also, though less common, colonize the lungs and causes pneumonia. This is the result of infected hospital breathing equipment. Symptoms for pneumonia include fever, chills, chest pain, rales, and cough. Prostatitis can occur as a result of Prostatitis mirabilis infection, causing fever, chills, and tender prostate in men. Proteus mirabilis infections can be treated with broad-spectrum penicillins or cephalosporins except in severe cases. It is not susceptible to nitrofurantoin or tetracycline and has experienced increased drug resistance to ampicillin, trimethoprim, and ciprofloxin. In cases with severe stone formation, surgery is necessary to remove the blockage. Proteus mirabilis is part of the normal flora of the gastrointestinal tract, and as a result the bacterium enters the urinary tract or infects medical equipment by the fecal route. Consequently, prevention includes good sanitation and hygiene, including proper sterilization of medical equipment. It is also suggested that patients not requiring catherization should not receive catherization, despite its convenience for the caretaker.

Providencia sturtii is a Gram negative, flagellated motile bacterium. It is an aerobic micro organism that is best grown at about 37° C. The bacterium is found in sewage and contaminated waters. It is also found in humans and animals. They are known to cause urinary tract infections, traveler's diarrhea and have been isolated from wounds caused by third degree burns. It can also reside in the gastrointestinal tract of humans and animals.

Salmonella Agona. Salmonella is a Gram negative, rod-shaped bacilli that can cause diarrheal illness in humans. Most persons infected with Salmonella develop diarrhea, fever, and abdominal cramps twelve to seventy-two hours after infection. Infection is usually diagnosed by culture of a stool sample. The illness usually lasts four to seven days. Although most people recover without treatment, severe infections may occur. Infants, elderly persons, and those with impaired immune systems are more likely than others to develop severe illness. When severe infection occurs, Salmonella may spread from the intestines to the bloodstream and then to other body sites and can cause death unless the person is treated promptly with antibiotics. Every year, approximately 35,000 cases of food poisoning caused by the salmonella bacterium are reported. Because most mild cases are never reported, the actual number is probably quite larger. Children are the most likely to get salmonellosis. Young children, older adults, and people with impaired immune systems are the most likely to have severe infections. They are microscopic living creatures that pass from the feces of people or animals to other people or other animals. The rod-shaped enterobacterium has a diameter of from about 0.5 to about 1.5 micrometers and a length of from 2.0 to about 5 micrometers.

Salmonella can survive for weeks outside a living body. They have been found in dried excrement after more than two and a half years. Salmonella are not destroyed by freezing. Ultraviolet radiation and heat accelerate their demise; they perish after being heated to 55° C. (131° F.) for one hour, or to 60° C. (14 ° F.) for half an hour. With poultry, cattle, and sheep frequently being agents of contamination, salmonella can be found in food, particularly meats and eggs. To protect against Salmonella infection, it is recommended that food be cooked for at least ten minutes at 75° C. (167° F.) so that the center of the food reaches this temperature. Salmonella bacteria can survive several weeks in a dry environment and several months in water; thus, they are frequently found in polluted water, contamination from the excrement of carrier animals being particularly important. Aquatic vertebrates, notably birds and reptiles, are important vectors of Salmonella.

Salmonella Poona has been associated with outbreaks in cantaloupes and turtles. Symptoms included nausea, vomiting, diarrhea, abdominal cramps, and fever; the duration of symptoms was three to twelve days.

Vibrio Cholerae 01 is one of two strains associated with epidemic cholerae. Vibrio 01, the bacterium that causes the disease cholera, controls virulence factor production and biofilm development in response to two extracellular quorum-sensing molecules, called autoinducers. The organism is a comma-shaped, Gram-negative aerobic bacillus whose size varies from one to three micrometers in length by 0.5-0.8 micrometers in diameter.

Vibrio s CMP6 is one of the few vibrio vulnificus strains of which the full genome has been sequenced.

Xylella fastidiosa, the pathogen causes Pierce's Disease, which has been a national issue for the viticulture industry. Xylella fastidiosa clogs a plant's xylem and effectively shuts down its ability to take in water and nutrients. The disease is spread naturally through the feeding activities of leafhopper vectors (Mizell, 1990).

Vibrio vulnificus M06 is the causative agent of life-threatening septicemia and severe wound infections.

Vibrio sp. can be isolated easily from water, soil, muscle tissue, blood, hemolymph, etc.

Vibrio parahaemolyticus P5 is one of the most widely used prey bacteria for isolation of halophilic BALOs.

In embodiments of the present invention, a preparation is provided comprising a therapeutically effective amount of a Bdellovibrio and like organism (BALO) and a bacteriophage combination introduced into a prey bacteria-infected subject.

The strains of BALO may comprise, for example, bacteriovorus phylotype III, bacteriovorax phylotype V, bacteriovorax phylotype IX, bacteriovorax phylotype X, bacteriovorax stolpii, bacteriovorax starrii, bdellovibrio bacteriovorax BD-610, bdellovibrio bacteriovorax HD100, peredibacter starrii, and bacteriolyticum stolpii. Pharmaceutically acceptable carriers may comprise, for example, sterile seawater, sterile physiological saline, sterile distilled water, and others known in the art. Bacteriophage may include, for example, CK2, 3a, ap3, A3/2, A10/A45, B9PP, A36, E13, E14, B9GP, 531, PM6, phage 1, VD13, phage 182, VD1884, λ phage, T even phages, T1 UV, T3, T5, T7, Esc-7-11, E920g, pt1 M13, MS2, P1, P2, Phi x 174, Phi 6, λvir, RB69, N4, 121Q, β4Q, HK243, BW-1, Haiti phage, I2-2, PR64FS, phage M, phage J, PR772, C-1, Phi 92, pilHα, Her252, Mu, H-19J, P1kc, R17, omega8, O103, K20, SS4, K30, O9-1, HK97, P1D, TC4, MB4, MS2, 13/3a, gh-1, epsilon15, phage x, phage 1-11 heidelberg, phage 16-19, phage 7-11, 9266Q, phage 2.5A, phage Jersey, phage Beccles, SasL4, Sas L6, phage O1, Vil, Vill, alpha3A, Beta, phage 16, phage 24, phage X29, Kappa, phage 4996, phage 57, phage e4, phage 45, phage 13, phage 14, phage 32, CP-T1, phage 493, VP1, VP11, VP12, VP6, KVP20, KVP40, Vf33, Phi 16, Phi HAWI-5, Phi PEL8C-1, V71A-6 NCIMB 41174, NCIMB 41175, NCIMB 41176, NCIMB 41177, NCIMB 41178, NCIMB 41179, and the like.

In an embodiment of the present invention, a composition is provided comprising a therapeutically effective amount of one or more strains of a Bdellovibrio and like organism (BALO) and one or more bacteriophage introduced in to a Bdellovibrio and like organism (BALO) prey bacteria-infected host subject. The BALO and the bacteriophage combination may exist, for example, in equal proportion or in various ratios or percentages.

In yet another embodiment of the present invention, a method of preparing a combination composition is provided comprising two or more Bdellovibrio and like organisms (BALO's) and two or more bacteriophage introduced into a susceptible prey bacteria-infected host subject or cell. The strains of BALO may, for example comprise Bdellovibrio bacteriovorus, Bacteriovorax stolpii, Bacteriovorax starrii, Bacteriovorax, Bateriolyticum stolpii, Peredibacter starrii Bacteriovorax litoralis, Bdellovibrio bacteriovorus HD 100, Bdellovibrio bacteriovorus BD-610, Bdellovibrio bacteriovorus Phylotype X, Bdellovibrio bacteriovorus Phylotype IX, Bdellovibrio bacteriovorus Phylotype V, Bdellovibrio bacteriovorus Phylotype III and the like. The starter bacteria can be a Gram negative bacteria susceptible to BALO and phages and/or attenuated. The viral strains may, for example, comprise CK2, 3a, ap3, A3/2, A10/A45, B9PP, A36, E13, E14, B9GP, 531, PM6, phage 1, VD13, phage 182, VD1884, A phage, T even phages, T1 UV, T3, T5, T7, Esc-7-11, E920g, pt1 M13, MS2, P1, P2, Phi x 174, Phi 6, λvir, RB69, N4, 121Q, β4Q, HK243, BW-1, Haiti phage, 12-2, PR64FS, phage M, phage J, PR772, C-1, Phi 92, pilHα, Her252, Mu, H-19J, P1kc, R17, omega8, O103, K20, SS4, K30, O9-1, HK97, P1D, TC4, MB4, MS2, 13/3a, gh-1, epsilon15, phage x, phage 1-11 heidelberg, phage 16-19, phage 7-11, 9266Q, phage 2.5A, phage Jersey, phage Beccles, SasL4, Sas L6, phage O1, Vil, Vill, alpha3A, Beta, phage 16, phage 24, phage X29, Kappa, phage 4996, phage 57, phage e4, phage 45, phage 13, phage 14, phage 32, CP-T1, phage 493, VP1, VP11, VP12, VP6, KVP20, KVP40, Vf33, Phi 16, Phi HAWI-5, Phi PEL8C-1, V71A-6 NCIMB 41174, NCIMB 41175, NCIMB 41176, NCIMB 41177, NCIMB 41178, NCIMB 41179, and the like.

Bacteriophage (Bacteria Viruses)

A bacteriophage is a virus that is parasitic in bacteria. A bacteriophage uses the bacterium's energy and processes to produce more phages until the bacterium is destroyed and the new phage particles are released to invade surrounding bacteria. At one time it was believed that the use of bacteriophage might be an effective way to treat bacterial infections, but it was thought that phage are quickly removed from the body and thus, some research in the area was abandoned. Recently, new interest has developed in the possible use of bacteriophage for treatment of bacterial infections and in prophylaxis. There is growing evidence that bacteriophage will be used in clinical medicine in the future.

Although different bacteriophages may contain different materials they all contain nucleic acid and protein. Depending upon the phage, the nucleic acid can be either DNA or RNA but not both and it can exist in various forms. The nucleic acids of phages often contain unusual or modified bases. These modified bases protect phage nucleic acid from nucleases that break down host nucleic acids during phage infection. The size of the nucleic acid varies depending upon the phage. The simplest phages only have enough nucleic acid to code for three to five average size gene products while the more complex phages may code for over one hundred gene products.

The number of different kinds of protein and the amount of each kind of protein in the phage particle will vary depending upon the phage. The simplest phage have many copies of only one or two different proteins while more complex phages may have many different kinds. The proteins function in infection and to protect the nucleic acid from nucleases in the environment. Bacteriophage may include, for example, CK2, 3a, ap3, A3/2. A10/A45, B9PP, A36, E13, E14, B9GP, 531, PM6, phage 1, VD13, phage 182, VD1884, λ phage, T even phages, T1 UV, T3, T5, T7, Esc-7-11, E920g, pt1 M13, MS2, P1, P2, Phi x 174, Phi 6, λvir, RB69, N4, 121Q, β4Q, HK243, BW-1, Haiti phage, 12-2, PR64FS, phage M, phage J, PR772, C-1, Phi 92, pilHα, Her252, Mu, H-19J, P1kc, R17, omega8, O103, K20, SS4, K30, O9-1, HK97, P1D, TC4, MB4, MS2, 13/3a, gh-1, epsilon15, phage x, phage 1-11 heidelberg, phage 16-19, phage 7-11, 9266Q, phage 2.5A, phage Jersey, phage Beccles, SasL4, Sas L6, phage O1, Vil, Vill, alpha3A, Beta, phage 16, phage 24, phage X29, Kappa, phage 4996, phage 57, phage e4, phage 45, phage 13, phage 14, phage 32, CP-T1, phage 493, VP1, VP11, VP12, VP6, KVP20, KVP40, Vf33, Phi 16, Phi HAWI-5, Phi PEL8C-1, V71A-6 NCIMB 41174, NCIMB 41175, NCIMB 41176, NCIMB 41177, NCIMB 41178, NCIMB 41179, and the like.

In embodiments, the methods, therapies, preparations and compositions of the instant invention have been performed to evaluate their individual and combined effect of combinations of Bdellovibrio and like organism (BALO) with varying percentages of bacteriophage in treating bacteria susceptible to strains of BALO and phage. One strain was tested in rabbits by the experimental ilial loop technique against Escherichia coli. The Bdellovibrio and like organism (BALO) strain Vibrio vulnificus in oysters was also tested for effacacy. Preliminary results suggest that when using phages or BALO, limited success was achieved. Based on modeling and laboratory results, we believe it more effective to use Bdellovibrio and like organism (BALO) in combination with phages to treat pathogens in infections. Synergistic effects have been realized and are shown in the examples of the instant application. Theoretically and experimentally, Bdellovibrio and like organism (BALO) are able to control a wide range of Gram negative bacteria whereas a cocktail of the bacteriophage strains were usually used to treat infection since their host range was narrower. We have found it to be very promising and efficacious to treat infections with bacteriophages and Bdellovibrio and like organism (BALO) in combination to achieve better results.

Bdellovibrio and like organism (BALO) and bacteriophages tested have not been shown to possess the required molecular machinery to infect humans indicating the safety of using the agents in combination to treat human bacterial diseases. If using these biological agents to decontaminant harmful bacteria before reaching the human body such as in foods for human consumption, presumably, the higher dose may achieve quicker effects. The highest dose of BALOs and phages we have tested were about 4×10⁸ PFU/mL, which were able to decrease by 5 logs the Vibrio vulnificus population within 40 hours in seawater. Those of skill in the art will determine the proper amounts, percentages, concentrations, and dosages using standards methods. However, a range of from about 4×10⁶ to about 4×10¹⁰ PFU/ml for both is reasonable.

### EXAMPLES

The following Examples are provided to illustrate certain aspects of the present invention and to aid those of skill in the art in practicing the invention. These Examples are in no way to be considered to limit the scope of the invention in any manner.

### Example 1

### Example of Routine Maintenance and Preparation of Prey Bacteria

Vibrio vulnificus strains were stored in LB-N containing 35% (vol/vol) glycerol at −70° C. Vibrio vulnificus wild type strains were grown on Luria-Bertani (LB) plates (Difco) containing 1.0% tryptone, 0.5% yeast extract, 1.0% NaCl and 1.5% (wt/vol) agar at 37° C. vibrio vulnificus FLA 042 were grow on LB-rif plates which was prepared by adding 50 μg/ml rifampicin in methanol to sterile LB agar at 47° C. Vibrio parahaemolyticus P-5 were grown on Sea Water Yeast Extract Agar (SWYE: 10 g/L Peptone, 3 g/L Yeast Extract, 15 g/L agar) (Kaneko and Colwell, 1973) at 30° C.

### Example 2

### Prey Suspensions to Eestablish the Microcosms or For Plating For Bacteriovorax Recovery

Prey suspensions were prepared by adding 5 mililiters of 70% artificial sea water (ASW) (Instant Ocean, Aquarium Systems, Inc., Mentor, Ohio) (pH 8, salinity 22 ppt.) to Luria-Bertani (LB) culture plates (Difco) containing an 18 hour culture of vibriovulnificus. The colonies on the plates were suspended in the liquid and the resulting prey suspension was transferred into a sterile tube for subsequent use. The viable prey bacterial counts were measured by spread plating in duplicate 0.1 mililiters of serial 10-fold diluted samples onto LB agar plates. The plates were incubated at 37° C. for two days and colony forming units (CFU) were counted and recorded. This or similar procedures known to those in the art was used to prepare other BALO prey suspensions.

### Example 3

### Example of Isolating Highly Efficient Predator Bacteriovorax Strains

Water samples were collected from sites in three different bodies of water located in Florida (USA): Dry Bar in Apalachicola Bay on three occasions, the eastern coast of the Gulf of Mexico, and Atlantic Ocean coastal waters off northern Florida. Immediately after being transported to the laboratory, water samples were mixed and filtered through a 0.8 micrometers (μm) filter to remove debris and larger organisms, for example, protists. Five hundred milliliters of the filtrate was dispensed into each of four 2 liter (L) Erlenmeyer flasks for the microcosm enrichment experiments. To complete the microcosms for the enrichment of Bacteriovorax, suspensions of target bacteria, Vibrio vulnificus and Vibrio parahaemolyticus were spiked as prey into the respective flasks described above to yield an optical density (OD) measurement of 0.7 at 600 nanometers (nm). This corresponds to approximately 5×10⁸ cells ml⁻¹ as enumerated by plate count on LB-rif agar plates [LB agar with 50 μg ml⁻¹ rifampicin]. The two control microcosms established to monitor the OD of the prey without interference from Bacteriovorax or other microorganisms consisted of equal volumes of prey in sterilized environmental water. The microcosm flasks were shaken at room temperature and monitored at 24 hour intervals through 120 hour by OD measurements (at 600 nm) in 48-well microtiter plates by an Absorbance Microplate Reader.

Isolation of Predominant Bacteriovorax Strains in Microcosms

Samples from the test microcosms were cultured for Bacteriovorax using the double agar overlay technique (Williams and Falkler, 1984). Before (pre-spike) and immediately (0 hours) after addition of the prey bacteria, 5 milliliters of sample were inoculated into Pp20 top agar tubes with 1 milliliter of prey bacteria (Vv or Vp) and plated onto large culture plates (150×15 mm). At subsequent time points, samples from the test microcosms were diluted by a series of 10-fold dilutions. One milliliter of the dilution was inoculated into 3.5 milliliters of molten Pp20 top agar tubes with 500 μl of prey. The contents of the tubes were mixed and overlaid onto Pp20 bottom agar plates. The plates were incubated at room temperature for up to 8 days. Plaque-forming units (PFUs) were counted and recorded. Plates with approximately 30 or less plaques were selected for further processing as these represented the predominant culturable Bacteriovorax OTU within the microcosm at the time of plating. Material from 80 to 100 percent of the plaques on these plates were collected with sterile micropipette tips and each inoculated, respectively, into a 1.5 mililiters eppendorf tube containing 50 microliters (μl) of autoclaved MiliQ water and stored at −20° C. for subsequent Polymerase Chain Reaction(PCR) analysis.

To determine the phylotype of highly efficient predator Bacteriovorax isolated, the tubes containing the selected Bacteriovorax plaques were boiled at 100° C. for 20 minutes. Ten micro-liters (μl) of the suspension was PCR amplified using Bacteriovorax specific primers, Bac-676F (5′-ATT TCG CAT GTA GGG GTA-3′) and Bac-1442R (5′-GCC ACG GCT TCA GGT AAG-3′) (Davidov, 2006) by puReTaq Ready-To-Go PCR Beads. All amplifications were performed under the following thermal conditions: initial denaturation at 95° C. for 3 minutes, followed by 34 cycles of 96° C. for 3 minutes, annealing at 55° C. for 1 minute, extension at 72° C. for 1 minute and a final extension at 72° C. for 7 minutes in an iCycler thermocycler. PCR products were analyzed by electrophoresis for amplicons of approximately 760 bp, purified with the QIAquick PCR-Purification Kit (QIAGEN) and sequenced with Bac-676F primer. DNA sequences and homology searches were analysed with the Basic Local Alignment Search Tool (BLAST) server from the National Center of Biotechnology Information (www.ncbi.nlm.nih.gov). Sequences were also analysed using the Chimera_Check, version 2.7 from the RDP-II Web site (Cole et al., 2003).

Bacteriovorax plate counts were obtained using the double agar overlay technique (Williams and Falkler 1984). Briefly, aliquots of samples were 10-fold serially diluted and inoculated into 3.5 mililiters molten Pp20 top agar (Pp20 bottom agar: 1 g L-1 Pepton, 15 g L-1 agar; Pp20 top agar: 1 g L-1 Peptone, 7 g L-1 agar) with 500 μl Vibrio vulnificus as prey. The contents of the tubes were mixed and overlaid onto Pp 20 bottom agar plates. The plates were incubated at room temperature (RT). The presence of Bacteriovorax plaques on the plates was monitored daily for a week and plaque forming units (PFUs) were counted and recorded within three days of their initial appearance.

### Example 4

### Example of Isolating Bacteriophage Strains Against Target Bacteria

The phage enrichment and isolation procedures were conducted on both estuarine mud sediment and oysters. When using oysters for the phage enrichment and isolation procedures, oysters were first scrubbed and washed under running deionized water. They were then shucked, and the oyster meat was removed for further use. Seawater at a salinity of 20 parts per trillion (ppt) was added to the oyster tissue at 1 mL/g of oyster tissue, and the mixture was homogenized. For all experiments using seawater, seawater was always used at 20 ppt. Fifty milliliters of either the homogenate or sediment mud contents was mixed with 50 mililiters of LB. The mixture was then inoculated with 1 mililiters of static overnight starter culture(s) consisting of vibrio vulnificus strain(s) of interest. The inoculated mixture was shaken overnight at 37° C.

After shaking overnight, the mixture was centrifuged at about 13,776×g for about 10 minutes at about 4° C. to remove bacterial debris, oyster homogenate, and mud sediment. The supernatant was then filtered through a 0.2 μm filter. The amplified phages in the filtrate were further amplified by mixing 1 mililiters of the filtrate with 1 mililiters of static overnight starter culture of vibrio vulnificus strain(s) of interest. This mixture of phage and bacteria was supplemented with 8 mililiters of LB and shaken overnight at about 37° C. If more than one vibrio vulnificus strain was used for the phage enrichment procedure, the filtrate was amplified separately in each of the vibrio vulnificus strains. After the culture was shaken overnight, it was centrifuged at 13,776×g for about 10 minutes at about 4° C., and the resulting supernatant was filtered through a 0.2 μm filter. Approximately 20 μl of the filtrate was streaked on LB plates, and about 4 mililiters of LB-SW soft agar inoculated with 1×107 CFU/ml of log phase vibrio vulnificus was poured onto LB-SW plates from the least concentrated area towards the most concentrated area of the streaked filtrate. The LB plate(s) was incubated overnight at about 37° C.

The next day, the clearest and most isolated plaques were picked using a Pasteur pipet. The agar plug was placed in 100 μl of BSG containing 10 μl of chloroform and was stored overnight at about 4° C. The mixture was then centrifuged at 13,776×g for 10 minutes at about 4° C. to remove bacterial debris and agar. The supernatant was used for a plaque purification step. The supernatant was streaked on a LB plate, and soft agar containing 1×10⁷ CFU/mL of log phase vibrio vulnificus was poured over the LB plate, as described above. The plate was incubated overnight at about 37° C., and the clearest and most isolated plaque was again picked using a Pasteur pipet. The agar plug was placed in 100 μl of BSG containing 10 μl of chloroform and stored overnight at a temperature of about 4° C. The next day, the mixture was centrifuged at 13,776×g for 10 minutes at about 4° C. to remove bacterial debris and agar. The resulting supernatant was stored at about 4° C. for the amplification procedure.

### Example 5

### Bacteriophage Amplification

Both broth and plate methods were employed for phage amplification. The majority of phages amplified efficiently using the broth amplification method, although certain phages amplified more efficiently using the plate method. Thus, the broth amplification technique, which necessitates less time for phage amplification, was utilized in certain embodiments unless, as detailed above, phages required the plate amplification technique for more efficient amplification.

Broth Phage Amplification Method

For the broth phage amplification method, 1 L of LB-N or LB-SW was inoculated with 5 mililiters of static overnight starter culture of vibrio vulnificus. The culture was shaken at about 37° C. until the culture reached an optical density corresponding to about 2×10⁷ CFU/mL. The culture was then infected with bacteriophage an MOI of 0.02 and shaken at about 37° C. until a change in the culture was observed from turbid to clear, corresponding to the phage induced lysis of the bacteria. One milliliter of chloroform was then added to the culture, which was shaken at about 37° C. for an additional 15 minutes to lyse any remaining bacteria. The culture was centrifuged at 13,776×g for about 10 minutes at 4° C. to remove bacterial debris, and the supernatant was stored at about 4° C. for the purification procedure.

Plate Phage Amplification Method

For the plate phage amplification method, 10 mililiters of LB-N or LB-SW broth was inoculated with a static overnight starter culture of vibrio vulnificus at a dilution of 1:20. The culture was shaken at about 37° C. until the culture reached a density of about 2×10⁸ CFU/mL, determined by OD600, and then about 4×107 CFU was combined with a volume of phage equivalent to an MOI of 0.5, and the tube was vortexed. After about a 10 minutes incubation period at room temperature, 4 mililiters of LB-SW soft agar was combined with the phage-bacteria mixture, vortexed, and poured onto a LB-SW plate. The soft agar overlay LB-SW plate was incubated overnight at about 37° C. The soft agar was removed using a sterile spatula, suspended in either 5 mililiters of BSG or seawater containing 20 μL of chloroform, and stored at about 4° C. After at least about 4 hours, the mixture was centrifuged at 13,776×g for about 10 minutes at about 4° C. to remove the soft agar and bacterial debris. The supernatant was stored at 4° C. for the purification procedure.

Purification of Phage

For purification of phage, 0.2 mililiters of 20 percent (wt/vol) polyethylene glycol (PEG) 8000, 2.5 M NaCl was added per mililiters of phage solution, vortexed, and stored overnight at about 4° C. The phage mixture was centrifuged at 13,776×g for about 10 minutes at about 4° C., and the resulting supernatant was discarded. To remove the remaining PEG in the phage suspension the mixture was centrifuged once more for approximately 1 minute at 13,776×g at 4° C., and the remaining supernatant was removed using a Pasteur pipet. The pellet was suspended in seawater and filtered through a 0.2 μm filter. The filtrate was stored at 4° C.

Quantification of Phage

For new phage solutions in which phage titers were unknown, the drop titer method was initially utilized to establish an approximate titer for each phage. The full plate titer method was utilized to establish a more accurate phage titer for each phage in the collection. Sterile seawater at 20 ppt was used for dilution of phage for all quantification assays. A culture of vibrio vulnificus was grown to about 2×10⁸ CFU/mL, and about 4×10⁷ CFU was infected with 100 μl of serially diluted phage and vortexed. After 10 minutes incubation at room temperature, about 4 mililiters of LB-N or LB-SW soft agar was added and vortexed. The resulting mixture was then poured over a LB-SW plate, and the plate was incubated overnight at about 37° C. The next day, the plaques were counted, and titer was calculated.

General Procedures

### Example 6

The Bacteriovorax present in the cultures were enumerated by quantitative real-time PCR (qPCR) (Zheng et al., 2008). Briefly, 1 mililiters samples were removed at 4 hour intervals and genomic DNA was extracted using the QlAamp DNA Mini kit (QIAGEN) with a final product of 100 μl eluted. Bacteriovorax specific primer set 519F (5′-CAGCAGCCGCGGTAATAC-3′) and 677R (5′-CGGATTTTACCCCTACATGC-3′) was used for quantification of the Bacteriovorax. qPCR analysis was performed by using the Bio-Rad CFX96 Real-Time PCR Detection System (Bio-Rad, Hercules, Calif., USA). The qPCR reaction mixtures (25 μl) were composed of 12.5 μl of iQ SYBR Green Supermix (Bio-Rad), 1 μl of each primer (5 pmol μl⁻¹) primer, 1 μl of sample DNA and 9.5 μl of MiliQ water. Thermal cycling conditions were: 2 minutes at 94° C., followed by 45 repeats of 30 sec at 94° C., 10 sec at 62° C. and 10 sec at 72° C. Each sample was measured in triplicate and negative controls (no template) were included. A 10-fold dilution series of plasmid containing a fragment of the Bacteriovorax 16S rRNA gene was used in the qPCR assay to construct the standard curve (correlation coefficient >0.99).

The combined effect of phages and BALOs in treating of Bdellovibrio prey bacterial infections were tested in seawater and two times in Luria Bertani broth. The temperatures we used were, for example, from about 25° C. to about 37° C. Test parameters including optical density readings, growth rate of the two predators and viable counts of the prey. Morphological features of BALO and phage infections were characterized by electron microscopy. Predator: prey ratios tested ranged from 10:1 to 1:100. BALOs and phages were inoculated in equal number initially and then the test cultures were monitored over about a 40 hour period with samples examined at selective time points.

To establish experimental cultures, equal numbers of Bacteriovorax and bacteriophages in respective suspensions were inoculated into a test microcosm containing Vibrio vulnificus suspended in 200 mililiters sterilized natural sea water at a predator: prey ratio of 10:1. Three control microcosms were established to monitor the growth of Vibrio vulnificus with Bacteriovorax and bacteriophages respectively or with no predators. Cultures were incubated at 27° C. on a shaker for 40 hours to monitor the population dynamics between the predator and prey and their respective abundances at selected time points. Test and control microcosms were monitored by measurements of OD values every 4 hours. Aliquots of samples were removed at 0, 12, 20 and 40 hours to obtain viable counts of Bacteriovorax, bacteriophages, and prey by plating methods described above.

To investigate and observe the combined effect of Bacteriovorax and bacteriophages by transmission electron microscopy, approximately 1×10 ⁸ PFU ml⁻¹ of Bacteriovorax cluster IX and bacteriophage CK2 were inoculated at predator-prey ratios of 1:1 into Luria-Bertani (LB) broth containing vibrio vulnificus at late logarithmic phase. The fresh prey culture was prepared by diluting the static overnight starter culture 1:20 into LB broth and shaking at 37° C. About 1.5 hour later, the shaking culture normally reached a cell density of approximately 2×10⁸ CFU ml⁻¹, corresponding to late logarithmic phase of growth. The prey bacteria were harvested by centrifugation at 13,776×g at room temperature for about 10 minutes. The resulting pellet was suspended in seawater prior to the addition of the predator. The mixture containing the two predators and their prey was shaken at about 37° C. Fifty mililiters of sample were removed after about 30 minutes, 1 hour, and 4 hours and fixed for electron microscopy examination according to Koval and Bayer, (1997) with modification. Briefly, samples were centrifuged for 30 minutes at 27,485×g, resuspended in 1 mililiters of 0.1 M sodium phosphate buffer (pH 7) and centrifuged for about 20 minutes at 20,142×g. The pellet was resuspended in 2 mililiters of 0.1 M cacodylate buffer containing 2 percent glutaraldehyde and 1 percent formaldehyde, both diluted from 25 percent (v/v) and 16 percent (v/v) stock solutions, respectively. After 1 hour at about 4° C. and centrifugation at 20,142×g for about 20 minutes, the pellet was overlaid with cacodylate buffer and an aliquot of sample was stained with uranyl acetate and examined with a Hitachi H-7600 transmission electron microscope.

The basic experiment described above will be adapted to evaluate the effects of experimental and environmental variables on the predation rate of BALOs and bacteriophages on test prey. The variables to be tested include: i) Initial predator-prey ratios of from about one to 100 to about ten to one, ii). Different prey bacteria (spike in single bacterium spp. as well as consortia of multiple species, iii). Various nutrient conditions, iv). Temperature range, v). Salinity range, and vi). pH range.

Effectiveness of Bacteriovorax and Phage in Reducing Vibrio in Oyster Model

Bacteriovorax and phage will be tested in an oyster model originally designed for evaluating bacteriophages (Martin, 2005) to investigate their effect on controlling Vibrio vulnificus in vivo. Briefly, live, fresh oysters from Dry Bar in Apalachicola Bay where the tested Bacteriovorax strain was originally isolated will be cleaned and placed in a pan with autoclaved seawater aerated with a Maxi-Jet 400 aquarium pump. After overnight acclimation and treatment with Rifampicin (Rif) (added to the seawater at a final concentration 50 μg/mL) to kill the natural bacterial flora and potentiate infection by the Vibrio vulnificus strain, the oysters will be experimentally infected with log phase Rif resistant Vibrio v. FLA042 or FLA077 at a final concentration of 1×10⁶ CFU/mL. Equal numbers of Bacteriovorax and phage ranging from 1×10⁷ to 1×10⁹ PFU/ml will be added individually and in combination to the oysters in the seawater for various periods of time. The oyster tissue will be harvested and homogenized and cultured to enumerate CFU of Vibrio vulnificus Vibrio vulnificus and PFU of Bacteriovorax and phage. The response of the two predators will be compared by analysis of variance (ANOVA) between groups. Real time PCR will be used as a nonculture method to enumerate Bacteriovorax and Vibrio vulnificus to validate plate count results.

Each of the strains will be grown and harvested as described in the above section. A purified suspension of each BALOs or bacteriophage isolate will be added to the water in the experimental holding tank for the rif treated oysters to achieve a final BALO/bacteriophage concentration of 10⁹ plaque-forming units ml⁻¹.

To examine potential detrimental effects of Bacteriovorax on mammals, mice models may, for example, be used be used. Groups of outbred ICR mice were injected intraperitoneally (i.p.) with Bacteriovorax and bacteriophage cultures suspended in phosphate-buffered saline at various PFU/mL. The interperitoneal route is generally the most sensitive in determining virulence of microorganisms because the bacteria are injected deep into the host into a site where they can multiply before an effective host response can be mounted. We initially inoculated the Bacteriovorax with a dose of about 1×10⁹ PFU. The heath of infected mice was monitored (i.e., death, scruffy fur, lethargy, or change in rectal temperature) for one week. At the end of one week, mice will be euthanized and the peritoneal cavity lavaged. The lavage fluid will be examined for Bacteriovorax and phages by plaquing. Bacteriovorax and bacteriophages in mixed suspensions were introduced orally into mice to examine their potential replication and pathogenicity in the intestinal tract. Mice will be starved of food and water for four hours. Bacteriovorax-phage cultures will be suspended in PBS to a final concentration of about 10¹⁰ PFU/mL. Mice will be fed 0.05 mL of 10 percent sodium bicarbonate to neutralize gastric acidity. Mice will then be fed 0.02 mL of BALO-phage mixed suspension, via a micropipet tip placed into the mouth. As above, the health of the mice will be monitored for one week, at which time they will be euthanized. The intestines will be removed, and the contents examined for HBALO by plaquing. A section of intestines will be fixed in 10 percent formalin and examined for histological damage by H&E staining. Samples of blood will also be examined for plaquing activity.

Bacteriovorax and bacteriophages tested have not been shown to possess the required molecular machinery to infect humans indicating the safety of using the agents in combination to treat human bacterial diseases. If using these biological agents to decontaminant harmful bacteria before reaching human body such as foods for human consumption, presumably, the higher dose may achieve quicker therapeutic effects. The highest dose of Bacteriovorax and phages we have tested were about 4×10⁸ PFU/mL, which were able to decrease by 5 logs of vibrio vulnificus population within 40 hours in seawater. In embodiments, a range of from about 1×10⁶ to about 1×10¹⁰ for both may, for example, be used.

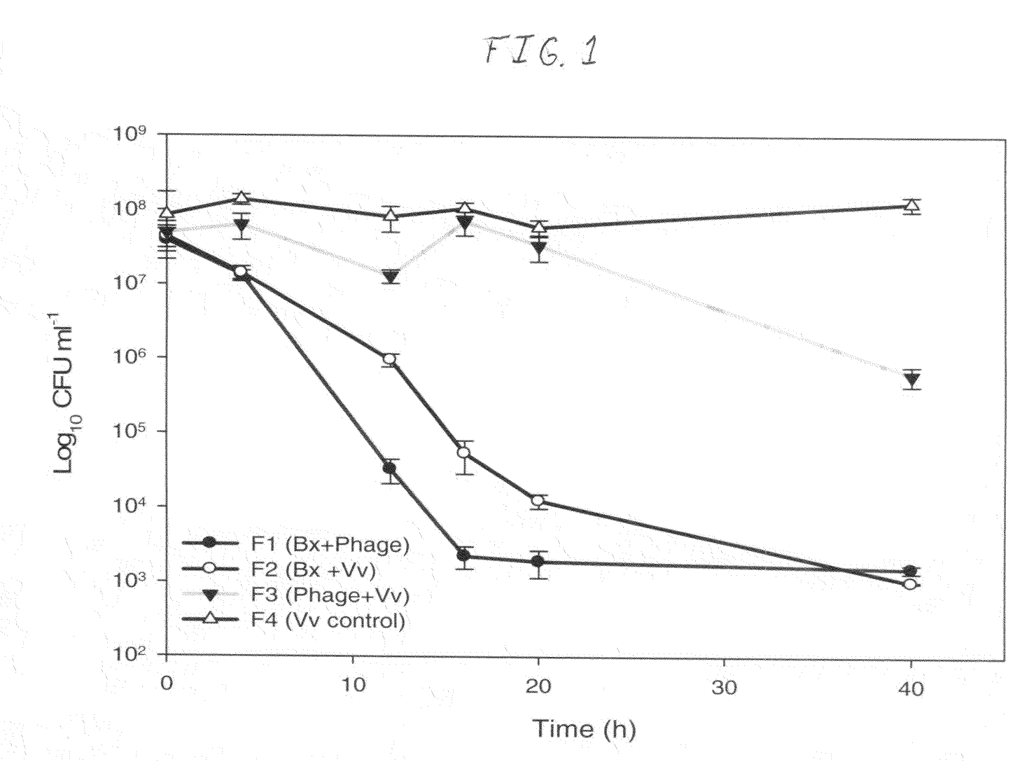

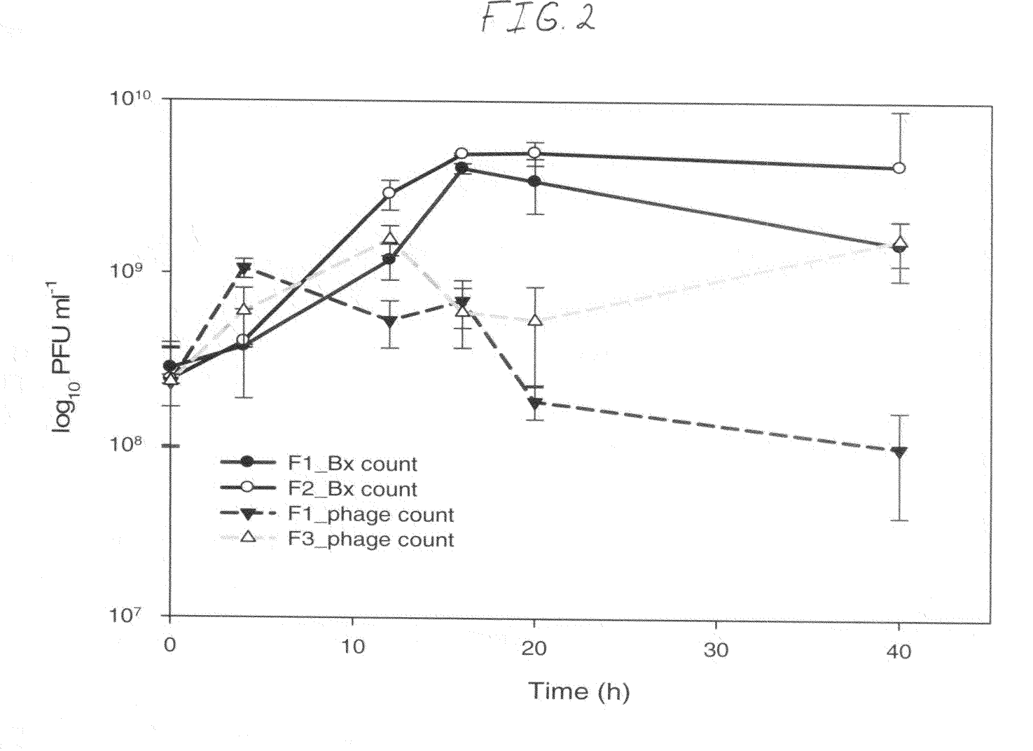

When Bacteriovorax and bacteriophages were inoculated into cultures of vibrio vulnificus, each was able to reduce the abundance of the prey significantly at 40 hour as measure by plate count (FIG. 1). The killing rate in microcosm Fl containing both Bacteriovorax and phage was significantly higher than those with a single predator after 4 hours. At 40 hours, the effect of Bacteriovorax and bacteriophage in combination was similar to that with Bacteriovorax alone, reducing the vibrio vulnificus by approximately 4.4 logs, whereas, with phage alone it was 1.9 logs (FIG. 2).

FIG. 1 Kinetics of the lysis of prey cells over time in test with Bacteriovorax (Bx)Bx and/or bacteriophages and control (with either predators or no predators). Error Bars are standard error from three independent experiments.

FIG. 2. Growth dynamics of Bacteriovorax and phage on prey over a 40 hour period as measured by plate counts. Broken lines designate phage count; solid lines represent Bacteriovorax counts. F1 is the flask with both Bacteriovorax and phage. F2 and F3 are the microcosms consisting of vibrio vulnificus with Bacteriovorax and phage respectively.

Bacteriovorax was the first and greatest responder to the prey, increasing 20-fold in plaque-forming units (PFU) to about 5×10⁹ PFU ml⁻¹ after about 12 hours incubation. The phage response was weak, as evidenced by little increase in the F1 flask and about a 5-fold increase in the F3 flask at about 40 hours. Both phage and Bacteriovorax abundance decreased in the Fl flask caused by low number of the remaining prey.

Results From Electron Microcopy

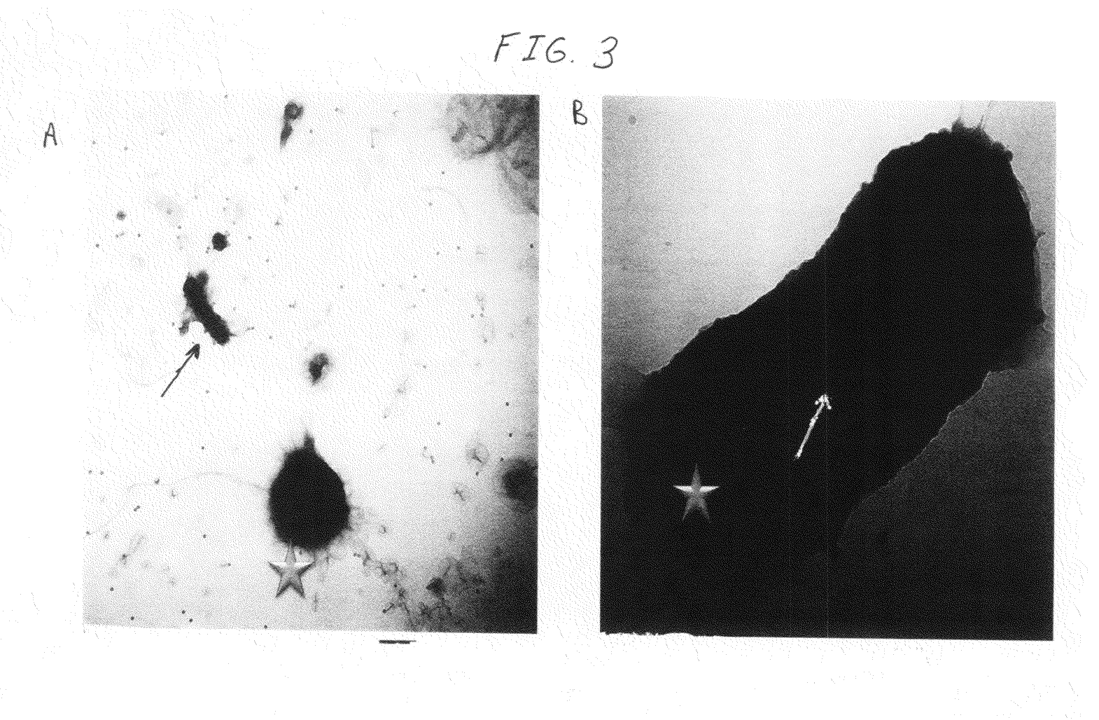

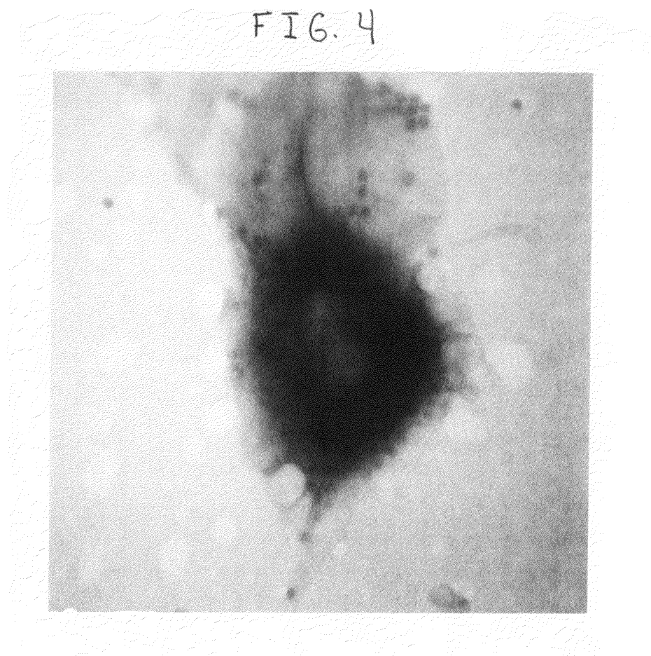

The results showed that Bacteriovorax and phage were able to infect vibrio vulnificus cells separately (FIG. 3A & B) and jointly (FIG. 4). Electron microscopy also confirmed that the Bacteriovorax, was effective at infecting the prey, vibrio vulnificus. Micrographs suggest that competition exists between Bacteriovorax and phages for the source of food as they both are able to prey on the same bacterium. Here we report a novel finding that Bacteriovorax and bacteriophages are able to infect and reside in a single prey cell resulting from dual infection.

FIG. 3. TEM micrographs showing the predation of bacteriophages and Bacteriovorax on prey vibrio vulnificus. The arrow in Figure A points to a prey cell infected by phage CK2. The star indicates a cell infected by Bacteriovorax to form a bdellobast (osmotically stable structure in which the prey cell becomes rounded) and no phage is seen inside. Figure B shows Bacteriovorax and phage both inside a vibrio vulnificus cell. Additional evidence showing Bacteriovorax and phage are able to infect the same cell.

While the invention has been described in connection with specific embodiments thereof, it will be understood that it is capable of further modifications and this application is intended to cover any variations, uses, or alterations of the invention. In general, the principles of the invention and including such departures from the present disclosure as come within known or customary practice within the art to which the invention pertains and as may be applied to the essential features hereinbefore set forth and as follows in the scope of the appended claims.

What is claimed is:

1. A method to decrease, treat or provide prophylactic support to a Bdellovibrio and like organism (BALO) Gram negative prey bacterial infection in a host, said method comprising administering a BALO and a bacteriophage combination to a prey bacterial infection in a ratio of from about ten to 1 to about 1 to one hundred, to inhibit, decrease, treat, or provide prophylactic support against the growth of a BALO Gram negative prey bacteria infected host, and wherein the BALO and bacteriophage combination are employed in a single combined preparation, and further wherein the Gram negative prey bacteria decreases by from about 1 log to from about 5 logs.

2. The method of claim 1 wherein the BALO Gram negative prey bacterial infection host is a plant, amphibian, animal, mammal, or a human.

3. The method of claim 2 wherein the BALO Gram negative prey bacteria host is a human.

4. The method of claim 1 wherein the BALO is selected from the group consisting of Bacteriovorax Phylotype III, Bacteriovorax Phylotype V, Bacteriovorax Phylotype IX, Bacteriovorax Phylotype X, Bdellovibrio bacteriovorus BD-610, Bdellovibrio bacteriovorus HD100, Peredibacter stank and Bacteriolyticum stolpii and wherein the Bdellovibrio and like organism (BALO), Gram-negative prey bacteria is selected from the group consisting of Acinetobacter calcoaceticus, Aeromonas hydrophila, Enterobacter aerogenes, Escherichia coil ML-35, Pseudomonas putida, Pseudomonas spp, Proteus mirabilis, Providencia stuartii, Salmonella, Salmonella Michigan, Salmonella Gaminola Salmonella Montbidea, Salmonella Poona, Vibrio 01, Vibrio s CMP6, Vibrio vulnificus, Vibrio vulnificus M06, Vibrio sp., and Vibrio parahaemolyticus P5, and further wherein the bacteriophage is selected from the group consisting of CK2, 3a, ap3, A3/2. A10/A45, B9PP, A36, E13, E14, B9GP, 531, PM6, phage 1, VD13, phage 182, VD1884, λ phage, T even phages, T1 UV, T3, T5, T7, Esc-7-11, E920g, pt1 M13, MS2, P1, P2, Phi x 174, Phi 6, λvir, RB69, N4, 121Q, β4Q, HK243, BW-1, Haiti phage, 12-2, PR64FS, phage M, phage J, PR772, C-1, Phi 92, pilHα, Her252, Mu, H-19J, P1kc, R17, omega8, O103, K20, SS4, K30, O9-1, HK97, P1D, TC4, MB4, MS2, 13/3a, gh-1, epsilon15, phage x, phage 1-11 heidelberg, phage 16-19, phage 7-11, 9266Q, phage 2.5A, phage Jersey, phage Beccles, SasL4, Sas L6, phage O1, Vil, Vill, alpha3A, Beta, phage 16, phage 24, phage X29, Kappa, phage 4996, phage 57, phage e4, phage 45, phage 13, phage 14, phage 32, CP-T1, phage 493, VP1, VP11, VP12, VP6, KVP20, KVP40, Vf33, Phi 16, Phi HAWI-5, Phi PEL8C-1, V71A-6 NCIMB 41174, NCIMB 41175, NCIMB 41176, NCIMB 41177, NCIMB 41178, NCIMB 41179, 152A-2, 152A-8, 152A-9 152A-10, 153A-5, 153A-7, 153A-8, 154A-8, 154A-9, 108A-9, 110A-7, 7-8a, CK-2, EJc, phage, 1a, 2a, 3a, 4a, 4b, AOIA-D, CKIA-B, and CKIF-G.

5. The method of claim 1, wherein said bacterial infection is an infection comprising a skin burn, skin infection, skin wound, a lung infection, an ocular infection, an ear infection, a sinus infection, a respiratory infection, a urinary tract infection, a infection from a medical device, an infection from a medical equipment or an infection from an implant.

6. The method of claim 5, wherein said infection is an ear infection.

7. The method of claim 5, wherein said infection is a urinary tract infection.

8. The method of claim 5, wherein said infection is a skin infection.

9. The method of claim 5, wherein said infection is a skin wound infection.

10. The method of claim 5, wherein said infection is a respiratory infection.

11. The method of claim 5, wherein said infection is a sinus infection.

12. The method of claim 5, wherein said infection is an ocular infection.

13. The method of claim 5, wherein said infection is caused by a medical device, medical equipment, or an implant.

14. The method of claim 5, wherein said BALO and bacteriophage are present in equal ratios.

15. A method to decrease, treat or provide prophylactic support to a BALO Gram negative prey bacterial infection selected from the group consisting of Acinetobacter calcoaceticus, Aeromonas hydrophila, Enterobacter aerogenes, Escherichia coli ML-35 Pseudomonas putida, Pseudomonas spp, Proteus mirabilis, Providencia stuartii, Salmonella, Salmonella Michigan, Salmonella Gaminola Salmonella Montbidea, Salmonella Poona, Vibrio 01, Vibrio s CMP6, Vibrio vulnificus, Vibrio sp., and Vibrio parahaemolyticus P5, said method comprising contacting said BALO Gram negative prey bacterial infection with a composition comprising a Bdellovibrio and like organism (BALD) and a bacteriophage equal ratio combination in an amount sufficient to inhibit , decrease, treat, or provide prophylactic support against the growth of said BALO Gram negative prey bacterial infection, and further wherein the Gram negative prey bacteria was reduced by from about 1.0 logs to about 5.0 logs.

16. The method of claim 15 wherein the Bdellovibrio and like organism (BALO) is selected from the group consisting of Bacteriovorax Phylotype III, Bacteriovorax Phylotype V, Bactetiovorax Phylotype IX, Bacteriovorax Phylotype X, Bdellovibrio bacteriovorus BD-610, Bdellovibrio bacteriovorus HD100, Peredibacter startii, and BacterioVicum stolpii, and wherein bacteriophage are selected from the group consisting of CK2, 3a, ap3, A3/2. A10/A45, B9PP, A36, E13, E14, B9GP, 531, PM6, phage 1, VD13, phage 182, VD1884, λ phage, T even phages, T1 UV, T3, T5, T7, Esc-7-11, E920g, pt1 M13, MS2, P1, P2, Phi x 174, Phi 6, λvir, RB69, N4, 121Q, β4Q, HK243, BW-1, Haiti phage, 12-2, PR64FS, phage M, phage J, PR772, C-1, Phi 92, pilHα, Her252, Mu, H-19J, P1kc, R17, omega8, O103, K20, SS4, K30, O9-1, HK97, P1D, TC4, MB4, MS2, 13/3a, gh-1, epsilon15, phage x, phage 1-11 heidelberg, phage 16-19, phage 7-11, 9266Q, phage 2.5A, phage Jersey, phage Beccles, SasL4, Sas L6, phage O1, Vil, Vill, alpha3A, Beta, phage 16, phage 24, phage X29, Kappa, phage 4996, phage 57, phage e4, phage 45, phage 13, phage 14, phage 32, CP-T1, phage 493, VP1, VP11, VP12, VP6, KVP20, KVP40, Vf33, Phi 16, Phi HAWI-5, Phi PEL8C-1, V71A-6 NCIMB 41174, NCIMB 41175, NCIMB 41176, NCIMB 41177, NCIMB 41178, NCIMB 41179, 152A-2, 152A-8, 152A-9 152A-10, 153A-5, 153A-7, 153A-8, 154A-8, 154A-9, 108A-9, 110A-7, 7-8a, CK-2, EJc, phage, 1a, 2a, 3a, 4a, 4b, AOIA-D, CKIA-B, and CKIF-G.

17. A method to decrease, treat or provide prophylactic support to a Bdellovibrio and like organism (BALO) Gram negative prey bacteria in a host, said prey bacteria is selected from the group consisting of Acinetobacter calcoaceticus, Aeromonas hydrophila, Enterobacter aerogenes, Escherichia coli ML-35, Pseudomonas putida, Pseudomonas spp, Proteus mirabilis, Providencia stuarlii, Salmonella, Salmonella Michigan, Salmonella Gaminola Salmonella Montbidea, Salmonella Poona, Vibrio 01, Vibrio s CMP6, Vibrio vulnificus, Vibrio sp., and Vibrio parahaemolyticus P5 comprising contacting the Gram negative prey bacteria with a Bdellovibrio and like organism (BALO) and a bacteriophage combination in an amount sufficient to inhibit, decrease, treat, or provide prophylactic support against the biological activity of a Gram negative prey bacteria host, and wherein said Bdellovibrio and like organism (BALO) is selected from the group consisting of Bacteriovorax Phylotype Ill, Bacteriovorax Phylotype V, Bacteriovorax Phylotype IX, Bacteriovorax Phylotype X, Bdellovibrio bacteriovorus BD-610, Bdellovibrio bacteriovorus HD 100, Peredibacter starrii, and Bacteriolyticum stolpii, and further wherein a bacteriophage is selected from the group consisting of CK2, 3a, ap3, A3/2. A10/A45, B9PP, A36, E13, E14, B9GP, 531, PM6, phage 1, VD13, phage 182, VD1884, A phage, T even phages, T1 UV, T3, T5, T7, Esc-7-11, E920g, pt1 M13, MS2, P1, P2, Phi x 174, Phi 6, λvir, RB69, N4, 121Q, β4Q, HK243, BW-1, Haiti phage, 12-2, PR64FS, phage M, phage J, PR772, C-1, Phi 92, pilHα, Her252, Mu, H-19J, P1kc, R17, omega8, O103, K20, SS4, K30, O9-1, HK97, P1D, TC4, MB4, MS2, 13/3a, gh-1, epsilon15, phage x, phage 1-11 heidelberg, phage 16-19, phage 7-11, 9266Q, phage 2.5A, phage Jersey, phage Beccles, SasL4, Sas L6, phage O1, Vil, Vill, alpha3A, Beta, phage 16, phage 24, phage X29, Kappa, phage 4996, phage 57, phage e4, phage 45, phage 13, phage 14, phage 32, CP-T1, phage 493, VP1, VP11, VP12, VP6, KVP20, KVP40, Vf33, Phi 16, Phi HAWI-5, Phi PEL8C-1, V71A-6 NCIMB 41174, NCIMB 41175, NCIMB 41176, NCIMB 41177, NCIMB 41178, NCIMB 41179, 152A-2, 152A-8, 152A-9 152A-10, 153A-5, 153A-7, 153A-8, 154A-8, 154A-9, 108A-9, 110A-7, 7-8a, CK-2, EJc, phage, 1a, 2a, 3a, 4a, 4b, AOIA-D, CKIA-B, and CKIF-G, and wherein the BALO and bacteriophage combination decreases about 2.0 logs to about 5.0 logs of the BALO prey bacterial population within forty hours of contacting the prey, and wherein the BALO and bacteriophage combination to said Gram negative prey bacteria ratio is from about ten to one to about one to one hundred.

18. A method of decreasing, treating or prophylaxis of a Bdellovibrio and like organism (BALO) Gram negative prey bacterial infection in a host cell, said method comprising administering a BALO and a bacteriophage combination to said prey bacterial infection at a temperature of from about 25° C. to about 37° C., wherein said prey bacterial infection is Vibrio vulnificus and wherein said BALO comprises Bacteriovorax, Peredibacter starrii, Bacteriolyticum stolpii, and Bdellovibrio bacteriovorus, and further wherein said BALO to prey bacterial infection in said host cell exists at a ratio of from about ten to one, to about one to one hundred.
